(19) United States
(12) Patent Application Publication
(10) Pub. No.: US 2022/0087661 A1
(43) Pub. Date: Mar. 24, 2022

(54) ROTATABLE SYRINGE DEVICE WITH SIDE CUTTING BIOPSY NEEDLE

(71) Applicant: Praxis Holding LLC, Tampa, FL (US)

(72) Inventors: John Steele Fisher, Belleair, FL (US); Nathaniel H. Pariseau, Tampa, FL (US); Wayne A. Noda, Mission Viejo, CA (US); Christopher M. Drake, Portsmouth, NH (US)

(21) Appl. No.: 17/545,038

(22) Filed: Dec. 8, 2021

Related U.S. Application Data
(63) Continuation-in-part of application No. 17/524,219, filed on Nov. 11, 2021.
Application No. 17/524,219 is a division of application No. 16/013,522, filed on Jun. 20, 2018, now Pat. No. 11,229,424.
(60) Provisional application No. 62/652,367, filed on Apr. 4, 2018.
(60) Provisional application No. 63/122,671, filed on Dec. 8, 2020.

(51) Int. Cl. A61B 10/02

(52) U.S. Cl. CPC A61B 10/0283; A61B 2010/045; A61B 2010/0208

(57) ABSTRACT

A syringe is coupled to a biopsy needle through a coupling structure that includes a motor-driven element such as a gear to rotate the needle. The needle can have a sharp beveled tip which, in cooperation with rotation of the needle, harvests tissue in vivo via rotation rather than multiple “sticks” into the patient.

### CROSS-REFERENCE TO RELATED APPLICATIONS

This nonprovisional application is a continuation in part of and claims priority to nonprovisional application Ser. No. 17/524,219, entitled “Rotatable Syringe Device,” filed Nov. 11, 2021 by the same inventor(s), which is a divisional of and claims priority to nonprovisional application Ser. No. 16/013,522, entitled “Rotatable Syringe System,” filed Jun. 20, 2018 by the same inventor(s), which claims priority to provisional application No. 62/652,367 filed Apr. 4, 2018 by the same inventor(s).

This nonprovisional application also claims priority to provisional application No. 63/122,671, entitled “Biopsy Needle with Cutting Structure and Related Method of Manufacture,” filed Dec. 8, 2020 by the same inventor(s).

### BACKGROUND OF THE INVENTION

### 1. Field of the Invention

The application relates generally to rotatable syringe systems, and more particularly to biopsy syringe systems with rotatable needles.

### 2. Brief Description of the Prior Art

Biopsy needles are often used to remove a cellular material to determine if a suspicious mass is malignant. If the mass is determined to be malignant, the cellular material is similarly used to identify the tumor-specific mutations that allow for personalized treatment.

Traditional biopsy needles, such as those disclosed in U.S. Pat. No. 9,084,465 B2 ('465 patent) fail to adequately collect enough cellular material on each pass, resulting in a need for multiple passes. However, requiring multiple passes during the procedure extends the overall length of the biopsy procedure and increases patient risk.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

As understood herein, it is often necessary to make multiple needle insertions in the patient to obtain sufficient tissue for analysis. Multiple “sticks” is a drawback for both patient comfort and clinical efficiency. Occasionally the need for further biopsy “sticks” is not realized until after the patient has left the medical establishment and a technician discovers that insufficient tissue has been harvested for analysis, requiring the patient to return for additional, uncomfortable tissue harvesting.

Accordingly, to avoid drawing tissue samples of insufficient cells that as a consequence requires repetition, a rotating needle is driven by a motor to harvest significantly higher yields of cellular material, reducing cost and patient discomfort.

In an aspect, a device includes an elongated needle having a cutting tip and a hollow interior. A syringe is coupled to the needle for rotation of the needle relative to the syringe, with an evacuatable tissue chamber being established at least in part by the hollow interior of the needle. A motor is coupled to the needle to rotate the needle while the tissue chamber is evacuated, and the needle is disposed adjacent tissue to facilitate drawing cells from the tissue into the tissue chamber.

In example embodiments, the syringe includes a distal end configured as a connector, the needle is engaged with a needle hub, and the syringe is coupled to the needle by a coupling comprising at least a mating connector for the needle hub and a mating connector for the syringe connector. The example syringe includes a barrel and a plunger slidably disposed in the barrel and movable to evacuate the tissue chamber, and a valve such as a slide valve or stopcock or other valve structure is operably coupled to the coupling to lock vacuum in the tissue chamber.

In non-limiting implementations, the coupling may include at least one hollow fitting engaged with the needle hub. The hollow fitting includes a body that may be configured as a luer fitting and a driven gear circumscribing the body and meshed with a drive gear coupled to the motor. At least one support assembly is engaged with the hollow fitting to rotatably support the hollow fitting. The support assembly is coupled to the connector of the distal end of the syringe, if desired via at least one luer fitting. The hollow fitting that is engaged with the needle hub may rotate against an O-ring engaged with the support assembly.

In some implementations, the motor defines an axis of rotation, the needle defines a longitudinal axis, and the axis of rotation of the motor is co-linear with the longitudinal axis of the needle. In other implementations, the motor defines an axis of rotation, the needle defines a longitudinal axis, and the axis of rotation of the motor is offset from with the longitudinal axis of the needle. In this implementation a belt may couple the motor to the needle to cause the needle to rotate under influence of the motor. Or, driven and drive gears may couple the motor to the needle to cause the needle to rotate under influence of the motor.

In some examples, the needle is no larger than twenty-five (235) gauge and may be 25 gauge or 27 gauge. The needle may rotate at a speed in the range of sixty (60) revolutions per minute (RPM) to three hundred fifty (350) RPM, inclusive.

In example implementations, the syringe can include a barrel and a plunger slidably disposed in the barrel and proximally movable to evacuate the tissue chamber, and a plunger lock can be mounted on a proximal portion of the barrel. At least one notch can be formed in the plunger, with at least a portion of the plunger lock riding against the plunger until the notch is juxtaposed with the portion of the plunger lock to cause the portion of the plunger lock to engage the notch to impede distal movement of the plunger. The plunger can be rotatable in the barrel to disengage the portion of the plunger lock from the notch.

In another aspect, a device includes a needle, a needle hub supporting the needle, and a rotatable fitting connected to the needle hub. The rotatable fitting includes a body and a driven gear circumscribing the body. A support assembly is rotatably engaged with the rotatable fitting, and a syringe is coupled to the support assembly by at least one coupling. A fluid passageway for fluid communication between an interior of the needle and the syringe is established by the needle hub, rotatable fitting, and support assembly such that the syringe is manipulable to evacuate the interior of the needle. A motor is coupled to a drive gear that in turn is meshed with the driven gear to cause the needle to rotate under influence of the motor while the interior of the needle is evacuated.

In another aspect, a method includes retracting a syringe plunger proximally relative to a barrel of the syringe to a first proximal position and closing a vacuum opening in the barrel. After closing the vacuum opening, the method includes retracting the syringe plunger proximally relative to the barrel of the syringe to a second proximal position to evacuate the barrel. The second proximal position is more proximal than the first proximal position. The method includes advancing a needle in fluid communication with the barrel of the syringe into an object to be sampled, energizing a motor coupled to the needle to rotate the needle, and opening the vacuum opening to cause portions of the object to be sucked into the needle as the needle rotates. The method then includes deenergizing the motor, releasing the vacuum, withdrawing the needle from the object, and advancing the plunger distally to expel the portions of the object from the needle.

In some embodiments, the biopsy device includes a biopsy needle having a main body extending between a proximal end and a distal end. A connector is located at the proximal end and is configured to operably engage the motor, such that the motor can rotate the biopsy needle. A cutting tip located at the distal end and a central longitudinal axis extends between the proximal and distal ends. In addition, a hollow interior extends through the main body and the connector, such that the hollow interior of the needle is in fluidic communication with the tissue collector.

Some embodiments of the biopsy needle include a first cutting aperture disposed through the main body. The first cutting aperture has a rectangular shape with a long end of the rectangular shape extending parallel to the central longitudinal axis of the biopsy needle. A second cutting aperture is also disposed through the main body. The second cutting aperture has a rectangular shape identical to the first cutting aperture with a long end of the rectangular shape extending parallel to the central longitudinal axis of the biopsy needle. Each of the first and second cutting apertures creates a channel with a central axis extending from an exterior surface of an interior surface of the main body, and the central axes of the channels are aligned. Furthermore, the first and second cutting apertures are longitudinally spaced in a proximal direction from the distal end of the main body, such that there is a continuous portion of the main body between the first and second cutting apertures and the distal end of the main body.

In some embodiments, the motor further includes a rotational axis that is co-linear with the central longitudinal axis of the biopsy needle. Some embodiments include a belt coupling the motor to the biopsy needle to cause the biopsy needle to rotate under influence of the motor. Some embodiments further comprise at least driven and drive gears coupling the motor to the biopsy needle to cause the biopsy needle to rotate under influence of the motor.

Some embodiments further include a valve in operable communication with a tissue evacuation passage extending between the biopsy needle and the tissue chamber, wherein the valve can lock vacuum in the tissue evacuation passage. In some embodiments, the valve is a slide valve. The slide valve has a slidable actuator configured to occlude the tissue evacuation passage when slid into a closed position.

Some embodiments further include a connector on the proximal end of the biopsy needle is a luer connector. In some embodiments, at least one hollow fitting engages a needle connector. The hollow fitting comprises a body and a driven gear circumscribing the body and meshed with a drive gear coupled to the motor. Some embodiments further include the hollow fitting engaged with a needle connector rotates against an O-ring engaged with the drive housing.

In some embodiments, the biopsy needle is no larger than twenty-five (25) gauge. In some embodiments, the first and second cutting apertures are diametrically opposed from each other about the main body of the biopsy needle.

In some embodiments, each of the cutting apertures are defined by a boundary circumscribing the aperture and the boundary is generally flush with an exterior surface of the main body. In some embodiments, each cutting aperture includes a beveled channel wall extending between the interior and an exterior surface of the main body of the biopsy needle to direct tissue into the interior of the biopsy needle. In some embodiments, each of the cutting apertures includes an outwardly, laterally extending flange relative to the central longitudinal axis of the needle.

In some embodiments, each cutting aperture is the same distance from the distal end of the biopsy needle. In some embodiments, the first cutting aperture is longitudinally spaced from the second cutting aperture, such that the two cutting apertures are at different distances from the distal end of the biopsy needle.

In some embodiments, a portion of the biopsy needle proximal to the of the cutting apertures includes a plurality of annular grooves circumscribing an exterior surface of the biopsy needle.

Some embodiments further include a third and a fourth cutting aperture disposed through the main body. The third cutting aperture has a rectangular shape with a long end of the rectangular shape extending parallel to the central longitudinal axis of the biopsy needle. The fourth cutting aperture has a rectangular shape identical to the first cutting aperture with a long end of the rectangular shape extending parallel to the central longitudinal axis of the biopsy needle. The third and fourth cutting apertures are longitudinally spaced in the proximal direction from the distal end of the main body, such that the continuous portion of the main body is between the third and fourth cutting apertures and the distal end of the main body. In addition, the third and fourth cutting apertures are diametrically opposed from each other about the main body of the biopsy needle and the third cutting aperture is circumferentially spaced from the first cutting aperture by generally 45 degrees.

Some embodiments further include a plurality of annular grooves proximate the cutting apertures. In some embodiments, the distal end of the biopsy needle includes a beveled tip.

The details of the present application, both as to its structure and operation, can best be understood in reference to the accompanying drawings, in which like reference numerals refer to like parts.

### BRIEF DESCRIPTION OF THE DRAWINGS

For a fuller understanding of the invention, reference should be made to the following detailed description, taken in connection with the accompanying drawings, in which:

FIG. 1 is a side elevational view of a rotatable syringe system consistent with present principles;

FIG. 2 is a front elevational view of the system shown in FIG. 1;

FIG. 3 is a side elevational view of a rotatable syringe system constructed in accordance with an alternate embodiment consistent with present principles;

FIG. 4 is a front elevational view of the system shown in FIG. 3;

FIG. 5 is a side elevational view of another alternate embodiment consistent with present principles;

FIG. 6 is a front elevational view of the system shown in FIG. 5;

FIG. 7 is a perspective view of a first embodiment of a motor-driven needle assembly, showing a vacuum valve configured as a slide valve;

FIG. 8 is a perspective view of a second embodiment of a motor-driven needle assembly, showing a vacuum valve configured as a stopcock;

FIG. 9 is an exploded perspective of the assembly of FIG. 7 with the drive housing removed to show components internal to the drive housing;

FIG. 10 is a side cut-away view of the needle assembly in FIG. 7 with the drive housing removed to show components internal to the drive housing;

FIG. 11 is a perspective view of the interior of the distal portion of the needle assembly of FIG. 10, showing the rotatable coupling with driven gear meshed with the motor drive gear; and

FIG. 12 is a perspective view similar to FIG. 11, with the rotatable coupling removed to show the support boss and O-ring;

FIG. 13 is a side cut-away view of the needle assembly in FIG. 8 with the drive housing removed to show components internal to the drive housing;

FIG. 14 is a flow chart of example use;

FIG. 15 is a side cut-away view of the needle assembly that in all essential respects is identical to FIGS. 10 and 13, except no vacuum valve or plunger lock are used;

FIGS. 16 and 17 are schematic side diagrams of the slide valve shown in FIGS. 7 and 9 in the open and closed configurations, respectively;

FIG. 18 is a schematic view of a plunger lock mechanism with distal components including the needle and related drive train not shown for simplicity;

FIG. 19 is a perspective view of an embodiment of a biopsy needle used for the collection of cellular materials within a biopsy area;

FIG. 20A is a side view of an embodiment of a retrieval section of a biopsy needle used to collect cellular materials with a first and second tooth design;

FIG. 20B is a perspective view of an embodiment of a retrieval section of a biopsy needle used to collect cellular materials with a first and second tooth design;

FIG. 21A is a side view of an embodiment of a retrieval section of a biopsy needle used to collect cellular material with a uniform tooth design;

FIG. 21B is a perspective view of an embodiment of a retrieval section of a biopsy needle used to collect cellular material with a uniform tooth design;

FIG. 22A is a cross-sectional view of an embodiment of a retrieval section of a biopsy needle used to collect cellular material having a plurality of cutting apertures disposed within the body of the elongated shaft;

FIG. 22B is a close-up view of an embodiment of a cutting aperture disposed within the body of the elongated shaft;

FIG. 23A is a side view of an embodiment of a retrieval section of a biopsy needle used to collect cellular material having a series of alternating conical protrusions and cutting apertures;

FIG. 23B is a perspective view of an embodiment of a retrieval section of a biopsy needle used to collect cellular material having a series of alternating conical protrusions and cutting apertures;

FIG. 23C is an elevation diagram of an embodiment of a conical protrusion in FIG. 23A;

FIG. 24A is a side view of an embodiment of a retrieval section of a biopsy needle having a series of diametrically opposed cutting apertures;

FIG. 24B is a perspective view of an embodiment of a retrieval section of a biopsy needle having a series of diametrically opposed cutting apertures of FIG. 24A;

FIG. 24C is a top view of an embodiment of a retrieval section of a biopsy needle having a series of diametrically opposed cutting apertures taken along line A-A in FIG. 24A;

FIG. 24D is a top view of an embodiment of a retrieval section of a biopsy needle having a series of cutting edges extending from the exterior surface of the retrieval section;

FIG. 25A is a side view of an embodiment of a retrieval section of a biopsy needle having a series of grooves disposed within the body of the elongated shaft;

FIG. 25B is a perspective view of an embodiment of a retrieval section of a biopsy needle having a series of grooves disposed within the body of the elongated shaft;

FIG. 26A is a side view of an embodiment of a retrieval section of a biopsy needle having foreign objects disposed on an outer surface of the biopsy needle;

FIG. 26B is a side view of an embodiment of a retrieval section of a biopsy needle having foreign objects disposed on an outer surface of the biopsy needle;

FIG. 27A is a top view of an embodiment of a retrieval section of a biopsy needle having an elliptically shaped bore opening and a cutting aperture disposed within the body of the elongated shaft;

FIG. 27B is a cross-sectional view of an embodiment of a retrieval section of a biopsy needle having an elliptically shaped bore opening and a cutting aperture disposed within the body of the elongated shaft taken along line B-B of FIG. 27C;

FIG. 27C is a perspective view of an embodiment of a retrieval section of a biopsy needle having an elliptically shaped bore opening and a cutting aperture disposed within the body of the elongated shaft;

FIG. 28A is a perspective view of an embodiment of a retrieval section of a biopsy needle having cutting apertures disposed within the body of the biopsy needle at the same distance from the terminal end of the retrieval section;

FIG. 28B is a perspective view of an embodiment of a retrieval section of a biopsy needle having cutting apertures disposed within the body of the biopsy needle at a different distance from the terminal end of the retrieval section;

FIG. 28C is a cross-sectional view of an embodiment of a retrieval section of a biopsy needle taken along line C-C of FIG. 28A having cutting apertures disposed within the body of the biopsy needle;

FIG. 29A is a side view of an embodiment of a biopsy needle;

FIG. 29B is a side view of Detail X in FIG. 29A;

FIG. 29C is a cross-sectional view of an embodiment of a retrieval section of a biopsy needle taken along line A-A of FIG. 29A;

FIG. 30A is a side view of an embodiment of a biopsy needle;

FIG. 30B is a side view of an embodiment of a biopsy needle;

FIG. 30C is a cross-sectional view of an embodiment of a retrieval section of a biopsy needle taken along line A-A of FIG. 30A;

FIG. 31A is a side view of an embodiment of a biopsy needle;

FIG. 31B is a side view of Detail B in FIG. 31A;

FIG. 32 is a cross-sectional view of the elongated shaft of the biopsy needle depicting a laser cutting a pair of cutting apertures within the body of the elongated shaft;

FIG. 33A is a perspective view of an embodiment of a biopsy needle in which the cutting apertures are each disposed the same distance from the retrieval section;

FIG. 33B is a perspective view of an embodiment of a biopsy needle in which the cutting apertures are disposed at different distances from the retrieval section; and

FIG. 33C is a side view of an embodiment of a biopsy needle in which the cutting apertures are disposed at different distances from the retrieval section.

### DETAILED DESCRIPTION OF THE INVENTION

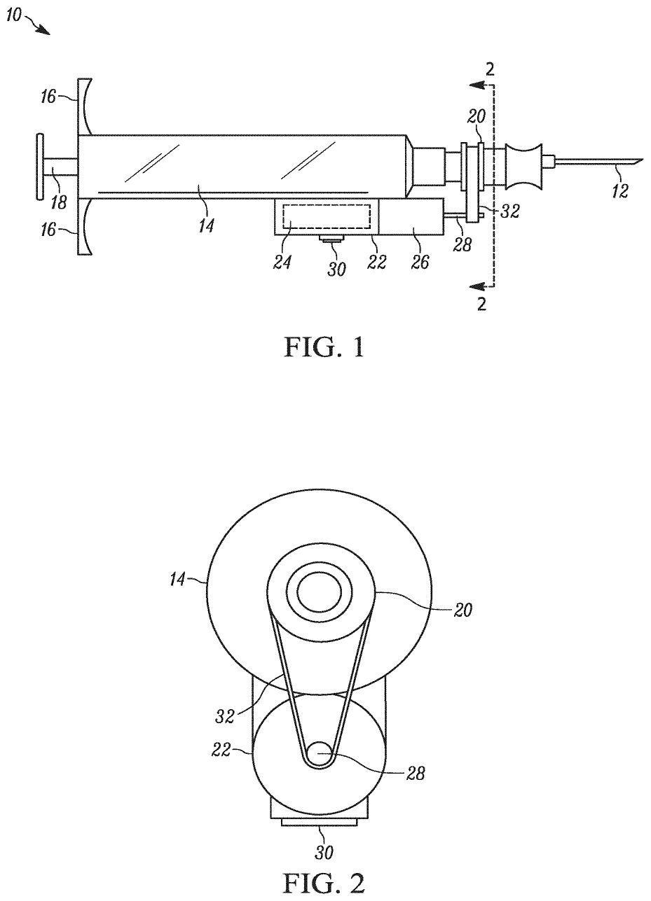

FIG. 1 illustrates a rotatable syringe system 10 that includes a barrel, a needle, a plunger, and a drive assembly. The rotatable syringe system 10 is for capturing tissue from within a patient and extracting from within the patient the captured tissue for analysis in a safe, convenient, and economical manner. In this context and as described more fully below, first provided is a barrel having a distal end and a proximal end. The proximal end of the barrel has laterally extending finger supports. A needle is provided having a distal end and a proximal end. The proximal end of the needle is operatively coupled to the distal end of the barrel. A plunger is provided having a distal end and a proximal end. The distal end of the plunger is coupled to the barrel. The proximal end of the plunger is adapted to be contacted by a thumb of a care giver with fingers on the finger supports. A drive assembly is also provided. The drive assembly includes a housing with a distal end and proximal end. A source of electrical potential is provided within the housing. A motor is operatively coupled to the needle. A switch on the housing functions to inactivate the motor and to activate the motor to rotate the needle.

Now describing the details of FIG. 1, the syringe system 10 includes a rotatable needle 12 for capturing tissue from within a patient and for extracting from the patient captured tissue for analysis. The needle 12 is coupled to a syringe that includes a barrel 14 formed in a cylindrical configuration and adapted to receive and support and dispense liquid such as medication.

The barrel 14 has a distal end and a proximal end. The proximal end has laterally extending finger supports 16. The needle 12 also has a distal end and a proximal end, and the distal end of the needle is formed with a point. The proximal end of the needle is operatively coupled to the distal end of the barrel.

The syringe also includes a plunger 18 with a distal end and a proximal end. The distal end of the plunger is located within the barrel 14 to advance liquid medication out of the barrel into and through the needle and into the patient. The proximal end of the plunger is adapted to be depressed by a thumb of a care giver with fingers on the finger supports. The barrel 14 and the needle 12 and the plunger 18 share a common central axis.

A pulley 20 is next provided. The pulley 20 is located between the proximal end of the needle 12 and the distal end of the barrel 14. The pulley 20 is attached to the needle for rotational movement therewith but with no axial movement of the needle. The pulley 20 is coupled to the barrel 14 for rotation independent of the barrel 14.

Next, a drive assembly is provided. The drive assembly includes a housing 22 in a cylindrical configuration with a distal end and proximal end. A source 24 of electrical potential is provided within and adjacent to the proximal end of the housing. A motor 26 is provided within the housing 22 adjacent the distal end of the housing 22. A drive shaft 28 is coupled to the motor 26. The drive shaft 28 extends forward from the motor 26 and the housing 22 to a location laterally spaced from the pulley 20. A switch 30 is provided to activate and inactivate the motor 26.

A belt 32 is trained around the drive shaft 28 and the pulley 20 to rotate the pulley 20 and the needle 12 for capturing tissue from within a patient and for extracting from the patient captured tissue for analysis.

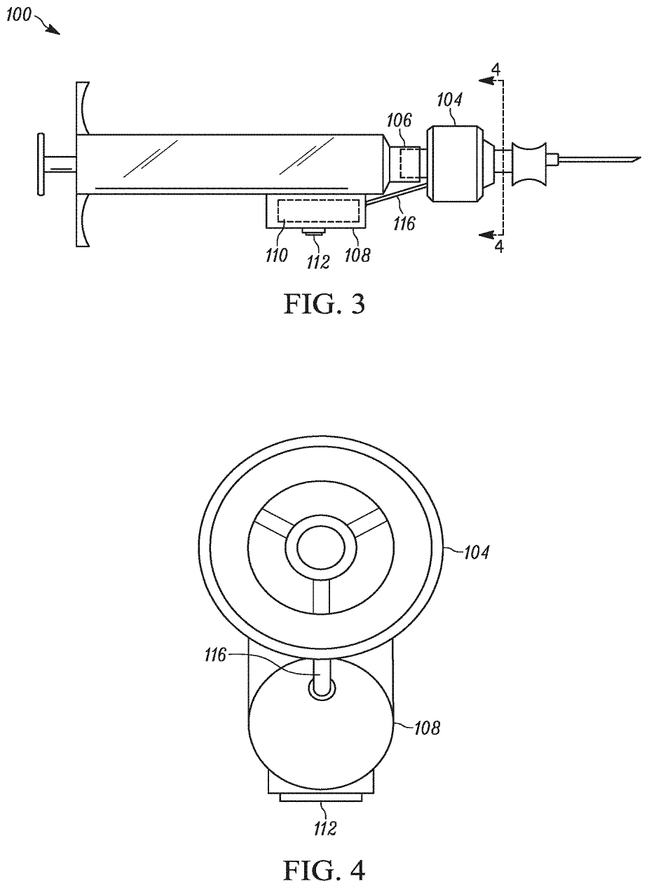

FIGS. 3-6 show alternate embodiments that may incorporate the structure shown in FIGS. 1 and 2, with the following exceptions.

FIGS. 3 and 4 illustrate an alternate system 100 with an in-line motor 104 located between the barrel of a syringe and a rotatable needle. The system 100 includes a housing 108 of a reduced size compared to the housing 22 shown in FIG. 1 and coupled to the barrel of the syringe. A source 110 of electrical potential is centrally located within the housing 108, and a switch 112 is centrally located on the housing to activate and deactivate the motor 104. A wire 116 couples the motor 104 and the source 110 of electrical potential.

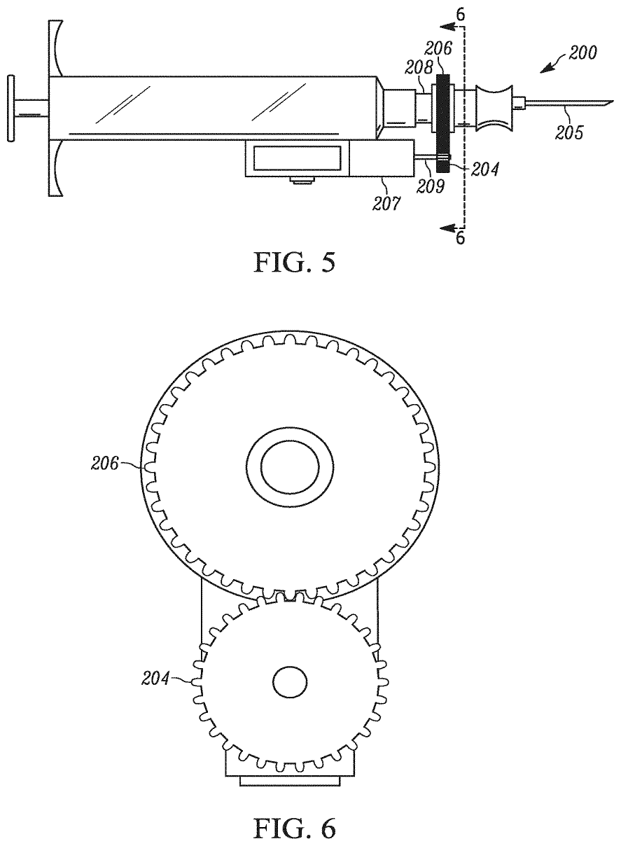

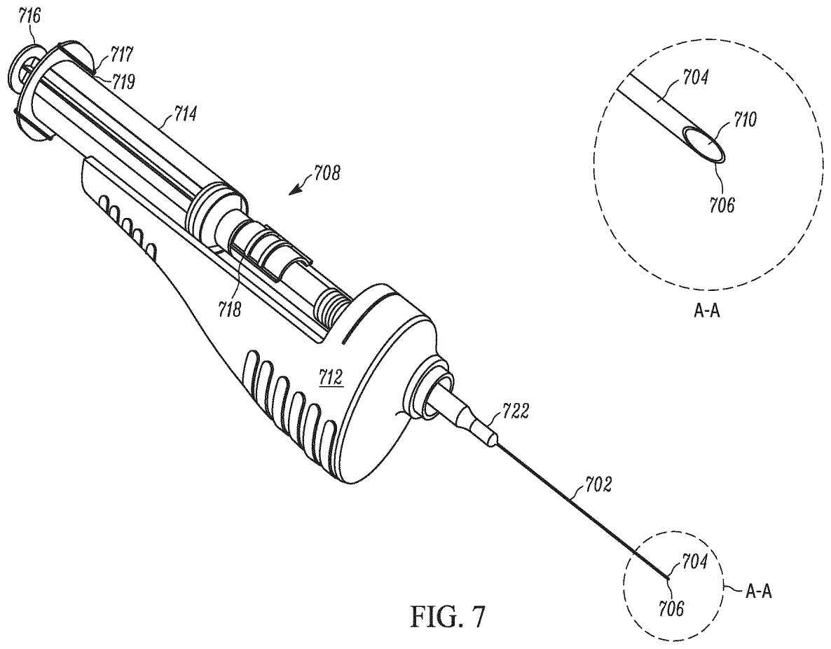

FIGS. 5 and 6 show yet another embodiment, a specific example of which is illustrated in FIG. 7 et seq. As shown, a driven gear 206 is coupled to needle 205 to rotate the needle 205. A motor is in a housing 207 with a drive shaft 209 extending forward of the housing 207 to a location laterally spaced from the driven gear 206. A drive gear 204 on the drive shaft 209 is meshed with the driven gear 206 for rotating the driven gear 206 and the needle 205.

In operation, any of the needles described herein can be placed at the edge of the tissue, such as a nodule, under ultrasound imaging or other imaging techniques such as ultrasound computed tomography (CT) guidance, magnetic resonance imaging (MRI) fluoroscopic guidance, and MRI imaging guidance. When the distal end of the plunger is withdrawn, a vacuum is created in the needle to withdraw tissue to be analyzed. The motor is energized with the switch on the syringe so that the procedure can be done with one hand. Once activated, the rotating needle is advanced in and out of the lesion, changing direction with each pass if desired. Once a sample is seen in the needle hub or syringe, the motor can be turned off and the needle removed. The sample can then be placed on slides for pathology evaluation. The motor can cause the needle to spin from 60 to 350 revolutions per minute (RPM), depending on what is determined to be the optimum speed. The needle may be advanced into the tissue sample for a period of 10 to 30 seconds or until a blood drop appears in the hub of the needle or syringe. During the fine needle aspirating (FNA), the rotating needle, preferably a 25 gauge needle, will be well visualized under ultrasound imaging. There will be for the most part only one pass into the nodule needed due to the highly effective cell shearing action of the rotating needle.

Now referring to FIGS. 7, a device 700 includes an elongated needle 702. The needle 702 may be a hollow metal hypodermic needle of a size of no more than twenty-five gauge (i.e., 25 gauge or greater gauge) with a cutting tip 704 as shown in detail A-A. The cutting tip 704 has a sharp cutting edge 706 that may be beveled as shown to facilitate cutting tissue when the needle 702 is advanced into tissue and rotated.

A syringe 708 is coupled to the needle 702 for rotation of the needle 702 relative to the syringe 708. An evacuatable tissue chamber 710 is established at least in part by the hollow interior of the needle 702. A motor, shown and described further below, is supported in a drive housing 712 and is coupled to the needle 702 to rotate the needle 702 while the tissue chamber 710 is evacuated and the needle 702 is disposed adjacent tissue to facilitate drawing cells from the tissue into the tissue chamber 710.

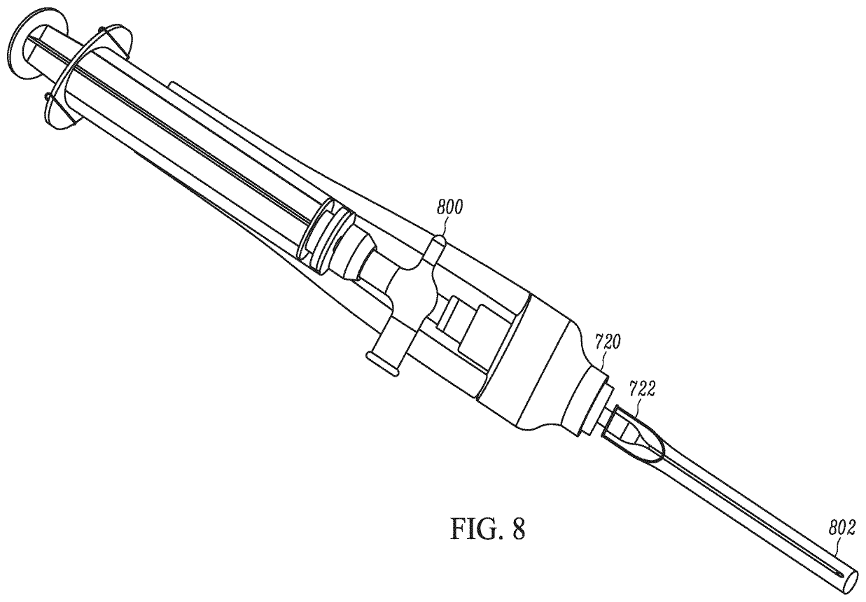

The syringe 708 typically includes a barrel 714 and a plunger 716 slidably disposed in the barrel 714 and movable to evacuate the tissue chamber 710. A valve such as a slide valve 718 (FIG. 7) or three-way stopcock 800 (FIG. 8) or other valve structure may optionally be provided to lock vacuum in the tissue chamber 710, although in some embodiments vacuum is established by appropriate manipulation of the syringe without the need for a valve. It is to be understood that the embodiment of FIG. 8 may in all other essential respects be identical to that of FIG. 7, with FIG. 8 additionally showing that the needle may be encased in a removable safety guard 802.

Completing the description of FIG. 7, in some embodiments a plunger lock mechanism 717 is engaged with the barrel 714, in this case with a proximal thumb flange 719 of the barrel 714, to engage one or more notches in the plunger 716 to impede advancing the plunger into the barrel (and for that matter to impede withdrawing the plunger out of the barrel). As more fully disclosed below, the plunger lock mechanism includes a stiff wire-like structure with a segment riding against the plunger 716 as the plunger is withdrawn proximally until the notch is juxtaposed with the segment to cause the segment to engage the notch under material bias. The plunger 716 can be rotatable in the barrel 714 to disengage the segment from the notch.

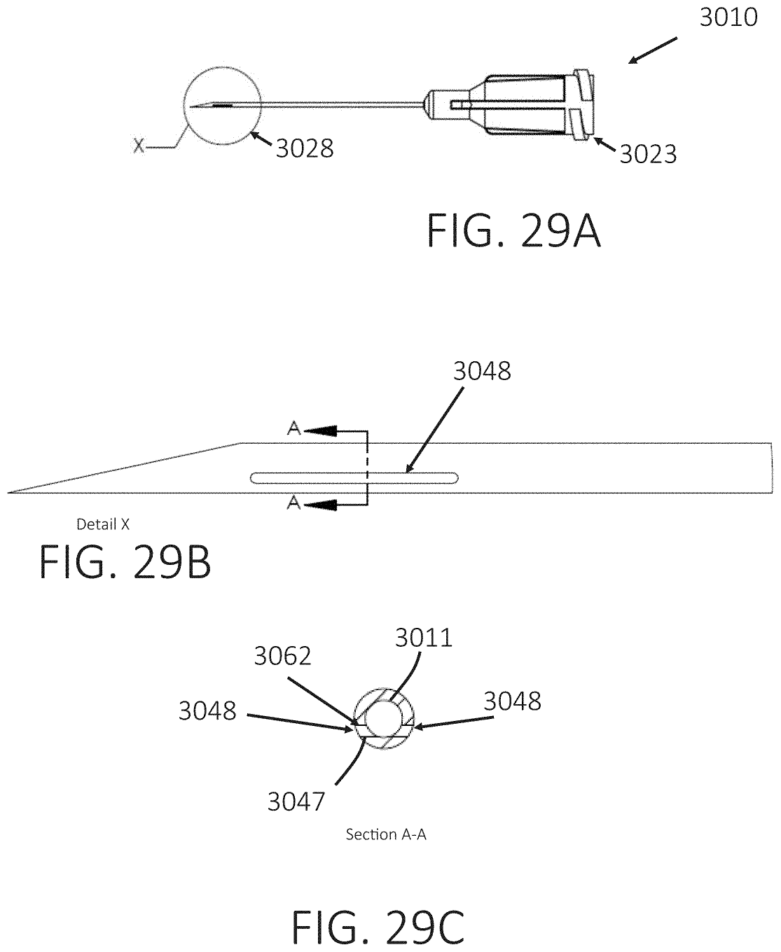

In example embodiments, as best shown in FIG. 8 the syringe can includes a distal end configured as a connector 720 (the distal end of the syringe 708 is obscured by the drive housing 712 in FIG. 7). The connector 720 may be configured as a luer fitting. As shown in FIGS. 7 and 8, the needle 702 is engaged with a needle hub 722, and the syringe 708 is coupled to the needle 702 by a coupling that includes at least the needle hub 722 and the connector 720. The needle hub 722 can be established by a hollow luer fitting such as a luer fitting (as an example see luer fitting 3023 in FIG. 29A).

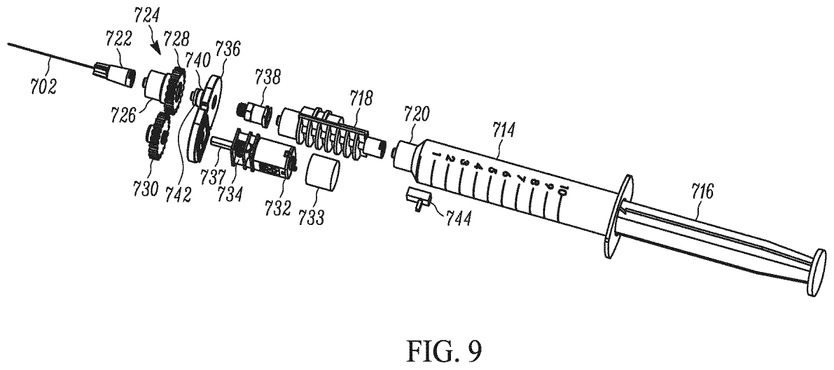

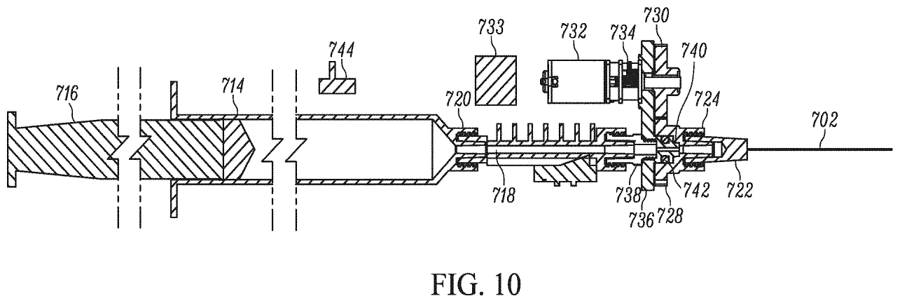

Indeed, and now referring to FIGS. 9 and 10, the above-mentioned coupling may include a hollow fitting 724 engaged with the needle hub 722. In the example shown, the hollow fitting 724 includes a body 726 that may be configured as a male luer fitting and a driven gear 728 circumscribing the body 726 and meshed with a drive gear 730 that is coupled to a small electric dc-powered motor 732 through a reduction gear assembly 734, which reduces rotational speed to be between sixty (60) revolutions per minute (RPM) to three hundred fifty (350) RPM, inclusive (which is therefore the rotational speed of the needle 702). The motor may be a six-volt DC gear motor operating at three VDC and powered by a battery 733 in the motor housing. These specifications are examples only. A Lithium or alkaline or other type of battery may be used, and the motor could operate at other voltages, e.g., 12 VDC operated by a 9 VDC battery.

A support assembly 736 may be engaged with the hollow fitting 724 to rotatably support the hollow fitting 724. Note that the output shaft 737 of the gear assembly 734 may extend through a hole of the support assembly 736 to connect to the drive gear 730, with the support assembly 734 radially supporting the output shaft 737 as the shaft spins.

The support assembly 736 is coupled to the connector 720 of the distal end of the syringe 708, if desired via at least one luer fitting 738 that may be, e.g., glued to the support assembly 736. When the slide valve 718 is included (or another valve such as a stopcock as set forth further below), the luer fitting 738 is connected to the distal end of the valve 718, which in turn is connected at its proximate end to the connector 720 of the syringe. The valve connectors may be configured as luer fittings. A continuous fluid passageway is formed from the tip of the needle 702 into the barrel 714 of the syringe by the train of components described above, with the valve 718 being operable to selectively occlude the fluid passageway to draw a vacuum in the system when the plunger is retracted proximally.

The hollow fitting 724 may rotate on a boss 740 of the support assembly 736, against an O-ring 740 that circumscribes the boss 740 to establish a fluid seal between the support assembly 736 and hollow fitting 724 during rotation.

As can be appreciated in reference to in FIGS. 9 and 10 and as mentioned above, a fluid passageway for fluid communication is established between the interior of the needle 702 and the syringe 708 by the needle hub 722, rotatable fitting 724, and support assembly 736 such that the syringe 708 is manipulable to evacuate the interior of the needle. The motor 732 that is coupled to the drive gear 730 that in turn is meshed with the driven gear 728 can be energized using a manipulable switch 744 such as a slide switch, toggle switch, moment switch, or other appropriate electrical switch to cause the needle 702 to rotate under influence of the motor 732 while the interior of the needle 702 is evacuated.

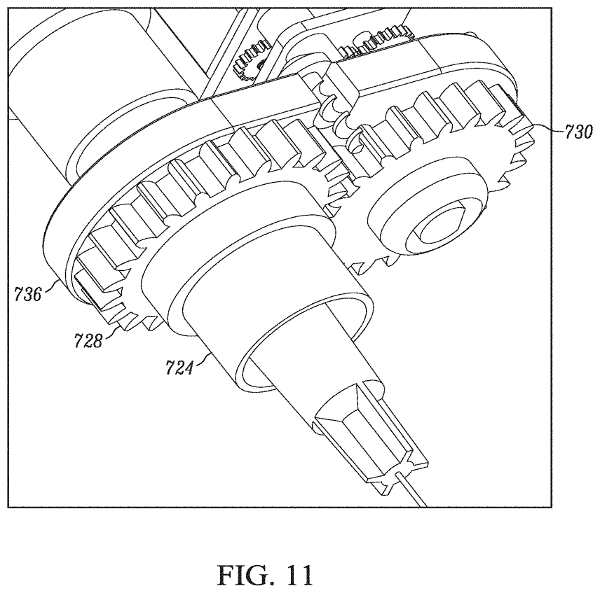

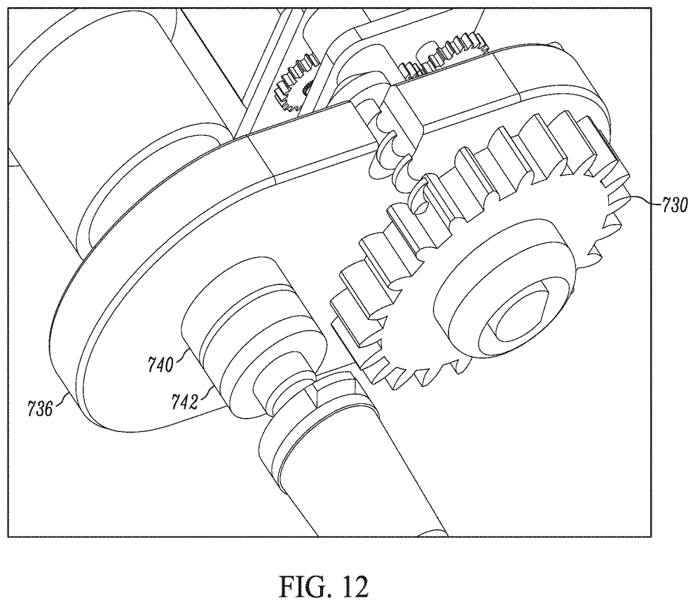

FIGS. 11 and 12 together provide further illustration of the drive gear 730, the driven gear 728 (FIG. 11 only), the rotatable fitting 724 (FIG. 11 only), the boss 740 and O-ring 742 (FIG. 12 only), and the support assembly 736.

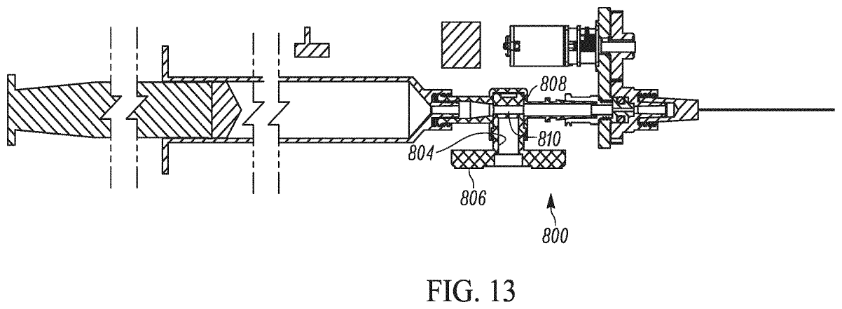

Now referring to FIG. 13, details of the stopcock 800, which may be a one-way stopcock or three-way stopcock, are shown. A cylindrical shaft 804 extending radially from a turnable handle 806 is rotationally engaged within a cylindrical housing 808. A cross hole 810 extends across the shaft 804 such that when the handle 806 is turned, the cross hole 810 is aligned with the proximal and distal outlets of the housing body 808 to allow flow from the needle to the syringe. Reversing the handle turn by 90 degrees seals off the cross hole 810 to block flow of material, fluids or air.

In operation, in some embodiments two to three drops of cells are sought to be obtained from tissue, to be dispensed as discussed below onto a microscope slide for analysis. This amount of sample typically can be held within the needle and hub alone, but to provide indication of adequate tissue harvest, enough tissue may be excised to fill not only the needle but also the fluid passageway between the syringe and needle and into the syringe, where tissue can be visualized and hence indicate sufficient sample has been obtained. With this in mind, the portion of the fluid passageway between the syringe and needle may be considered to be “dead space” which preferably is minimized in volume by making the diameter of the fluid passageway as small as practicable, since the fluid passageway must be filled with sample material before the caregiver sees anything in the syringe.

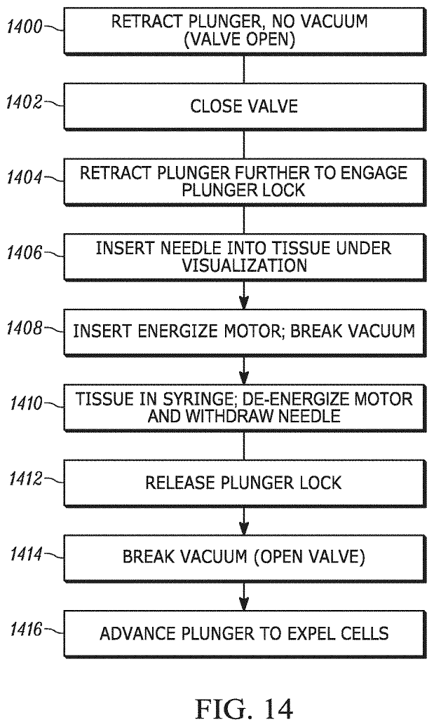

FIG. 14 is a flow chart of example use. At block 1400, the plunger is retracted, e.g., two to three milliliters, without creating a vacuum. When a vacuum valve is used, this is done by opening the valve to allow air into the barrel. After initial plunger retraction, the vacuum valve is closed at block 1402 and then at block 1404 the plunger is retracted further in the proximal direction until the plunger lock 1300 engages the plunger as described above. This creates a vacuum in the syringe.

At block 1406 the needle is next advanced into the target tissue. This procedure preferably is done in conjunction with ultrasound imaging. The ultrasound probe is held with one hand to image where the needle is and the target tissue.

Proceeding to block 1408, the motor is energized using, e.g., a slide switch, a moment switch, or other activating element. Vacuum in the syringe is broken by, e.g., opening the vacuum valve, which causes cells from the tissue to be sucked into the needle through the cutting tip with the needle constantly rotating the entire time. When tissue is visualized in the needle hub or syringe, indicating sufficient harvest, the motor is deenergized at block 1410, the vacuum is blocked, and the needle withdrawn from the patient.

Moving to block 1412, the plunger lock is released. Opening the vacuum valve without releasing the plunger should be avoided, as this will result in all the cells surging up into the syringe. When the plunger lock is released it will return to the relaxed position it assumed at block 1400, with some air, e.g., two to three milliliters, remaining in the barrel. At this point, at block 1414 the vacuum valve is opened. Moving to block 1416, the plunger is advanced distally to expel the cells out of the tip of the needle, typically onto a microscope slide for analysis. Note that owing to the small needle gauge in some embodiments, tissue cores are not harvested, only cells that are scraped from the tissue by the rotating needle.

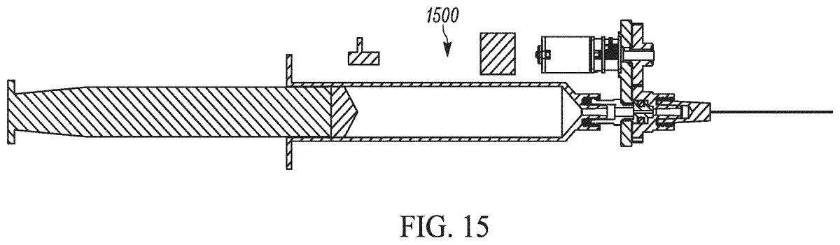

While FIG. 14 illustrates details of an example method, another method attendant to FIG. 15 below includes retracting the plunger slightly, advancing the needle under visualization such as ultrasound guidance to the target tissue or lesion, actuating the motor to rotate needle, retracting the plunger to create vacuum in the needle while the motor is spinning, releasing the plunger, deactivating the motor, retracting the needle from the patient, and dispensing the cells on a slide.

FIG. 15 shows an embodiment 1500 that in all essential respects is identical to those shown in FIGS. 7 and 8 and supporting FIGS., except that the vacuum valve and plunger lock are omitted, with different plunger manipulations used to perform use steps. Specifically, as mentioned above the procedure may be commenced with the plunger partially retracted, which provides enough plunger travel to create vacuum when needed and leaving some air volume in storage to dispense the cells. With the plunger partially retracted (essentially, step 1400 in FIG. 14), the needle is advanced into the tissue (essentially, step 1406 in FIG. 14), and the motor is energized to spin the needle to excise tissue (essentially, part of step 1408 in FIG. 14). At the same time, the plunger is retracted further proximally while the needle is spinning to draw tissue into the needle. Step 1410 in FIG. 14 is then performed to stop the motor and withdraw the needle, and then the process proceeds directly to step 1416 to expel tissue from the needle by advancing the plunger into the barrel of the syringe.

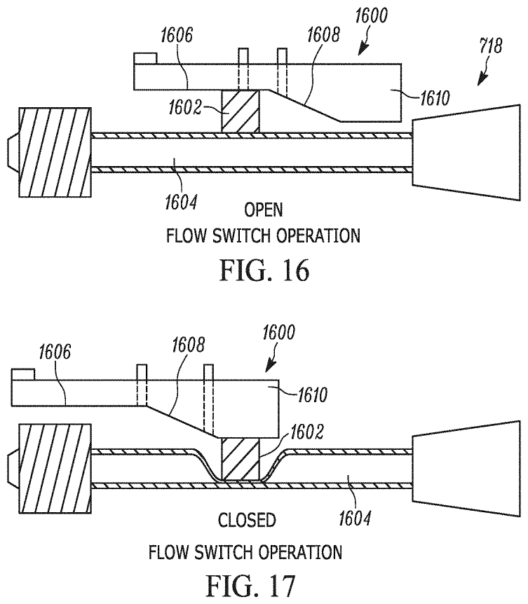

FIGS. 16 and 17 show example details of the slide valve 718. A slide 1600 can move longitudinally relative to the syringe to an open configuration (FIG. 16), in which a rigid compression block 1602 is disposed between a tube 1604 and a thinner section 1606 of the slide 1600 to allow the tube 1604, which like other tubes herein may be resilient, to remain open under material bias. The slide 1600 can move to a closed configuration (FIG. 17). When moving from the open to the closed configurations, a beveled section 1608 of the slide 1600 rides against the compression block 1602 to press the compression block 1602 against the tube 1604, a thick portion 1610 of the slide 1600 eventually reaching the compression block 1602 to completely occlude the tube 1604 in the closed configuration of FIG. 17.

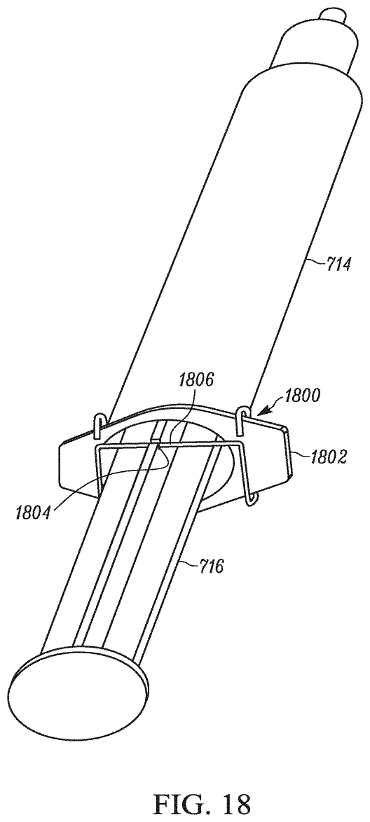

Now referring to FIG. 18, the plunger 716 of the syringe 708 that is slidably disposed in the barrel 714 is proximally movable to evacuate the tissue chamber. A stiff wire-like plunger lock 1800 can be mounted on a proximal portion 1802 of the barrel 714, in this case, a flat thumb surface. One or more notches 1804 can be formed in the plunger 716, with at least a segment 1806 of the plunger lock 1800 riding against the plunger 716 as the plunger is withdrawn proximally until the notch 1804 is juxtaposed with the segment 1806 of the plunger lock 1800 to cause the segment 1806 of the plunger lock 1800 to engage the notch 1804 to impede distal movement of the plunger 716. The plunger 716 can be rotatable in the barrel 714 to disengage the segment 1806 of the plunger lock 1800 from the notch 1804.

In some examples, a flexible shaft design may implement the rotational train of elements above and advanced down a bronchoscope or other endoscope. The needle may be larger than twenty-five gauge if desired to harvest tissue cores for biopsies.

Some embodiments of the present invention include a biopsy needle that dramatically increases cellular material (i.e., cells) yield per pass. By collecting more cellular material per pass, the biopsy procedure requires fewer passes and is completed in shorter periods of time over conventional biopsy needles.

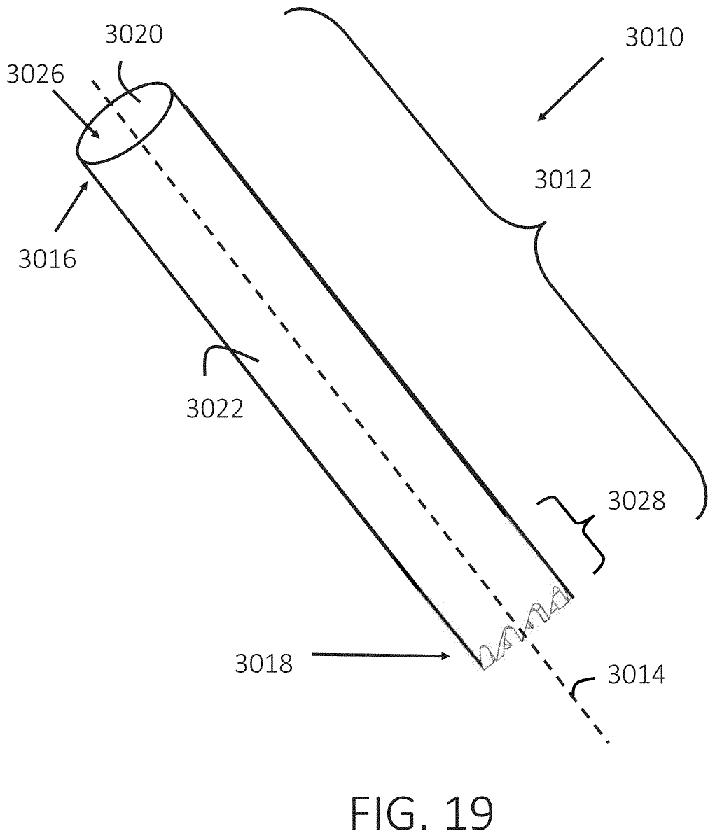

Referring now to FIGS. 19-33, biopsy needle 3010 is configured to penetrate one or more layers of tissues to obtain a sample of cellular material (such as cells or fluids) from within a target biopsy area. The cellular material is then analyzed to diagnose a medical condition or to rule out a disease. Biopsy needle 3010 is typically constructed of medical grade stainless nitinol, steel, or carbon steel; however, it is appreciated that biopsy needle 3010 may be constructed from other metals, polymers, carbon fiber, plastics, resins, composites, or any other biocompatible materials, which are pharmacologically inert.

Generally, biopsy needle 3010 comprises elongated shaft 3012 extending along central longitudinal axis 3014 from proximal end 3016 to distal end 3018. Elongated shaft 3012 includes internal surface 3020, external surface 3022, and body 3024 extending between internal surface 3020 and external surface 3022 of elongated shaft 3012. Moreover, internal surface 3020 of elongated shaft 3012 defines bore 3026, such that elongated shaft 3012 is hollow to facilitate the collection of cellular material from within the biopsy area.

Specifically, upon insertion of biopsy needle 3010 within a patient, the biopsy needle is manipulated (e.g., rotated and/or translated about its central longitudinal axis 3014) to enable the collection of cellular material and fluid. Once the cellular material is dislodged via the manipulation of biopsy needle 3010, the cellular material flows within bore 3026 from distal end 3018 to proximal end 3016 of biopsy needle 3010 and is collected within a collection reservoir (e.g., syringe or other devices) in mechanical communication with proximal end 3016 of biopsy needle 3010. Furthermore, distal end 3018 of biopsy needle 3010 includes retrieval section 3028 configured to scrape, tear, bump, grind, cut, sheer, hammer, or slash portions of intact cellular material located within the biopsy area to facilitate their collection within the collection reservoir through bore 3026.

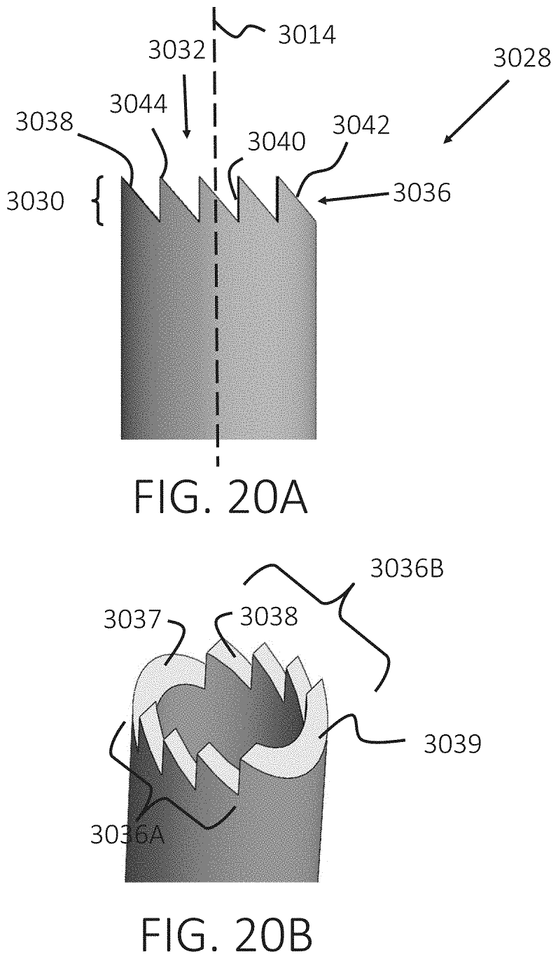

FIGS. 20A and 20B depict an embodiment of retrieval section 3028 disposed at distal end 3018 of biopsy needle 3010. As shown, retrieval section 3028 includes cutting edge 3030 disposed at leading-edge 3032 of retrieval section 3028. Cutting edge 3030 operably engages with the surrounding tissues to sheer off and dislodge cellular material from within the biopsy area during the biopsy procedure. In such embodiments, cutting edge 3030 includes alternating cutting designs disposed about a circumference of retrieval section 3028. In particular, a first cutting design and a second cutting design alternate to provide for the efficient capture of large portions of intact cellular material from within the biopsy area.

Cutting edge 3030 includes a first cutting design having a plurality of teeth 3036. Each tooth 3038 comprises face 3040, back 3042, and point 3044. A neutral rake angle of 0 degrees (i.e., rake angle being perpendicular to the direction of cut) is shown. The rake angle determines the angle of the cutting face 3040 of each tooth 3038. Moreover, having a rake angle of 0 degrees results in a vertical tooth 3038 that cuts faster and more aggressively. Furthermore, each tooth 3038 of cutting edge 3030 has a fleam angle (or bevel angle) of 0 degrees. In particular, the fleam is the angle across face 3040 of tooth 3038. The fleam permits each tooth 3038 to perform a tip-cut action—chiseling off cellular material as biopsy needle 3010 is manipulated and rotated about central longitudinal axis 3014.

In some embodiments, as depicted in FIG. 20B, cutting edge 3030 includes a single elongated tooth 3039, extending about leading-edge 3032 of retrieval section 3028 between groups of diametrically opposed teeth. Each tooth in groups 3036A and 3036B are longitudinally spaced in a distal direction moving from tooth 3039 to seat 3037, which is generally located in a diametrically opposed relation to tooth 3039. The rake and fleam angles of tooth 3039 and or the teeth in first plurality of teeth 3036A and second plurality of teeth 3036B may be the same or similar to those disclosed in relation to teeth depicted in FIG. 20A.

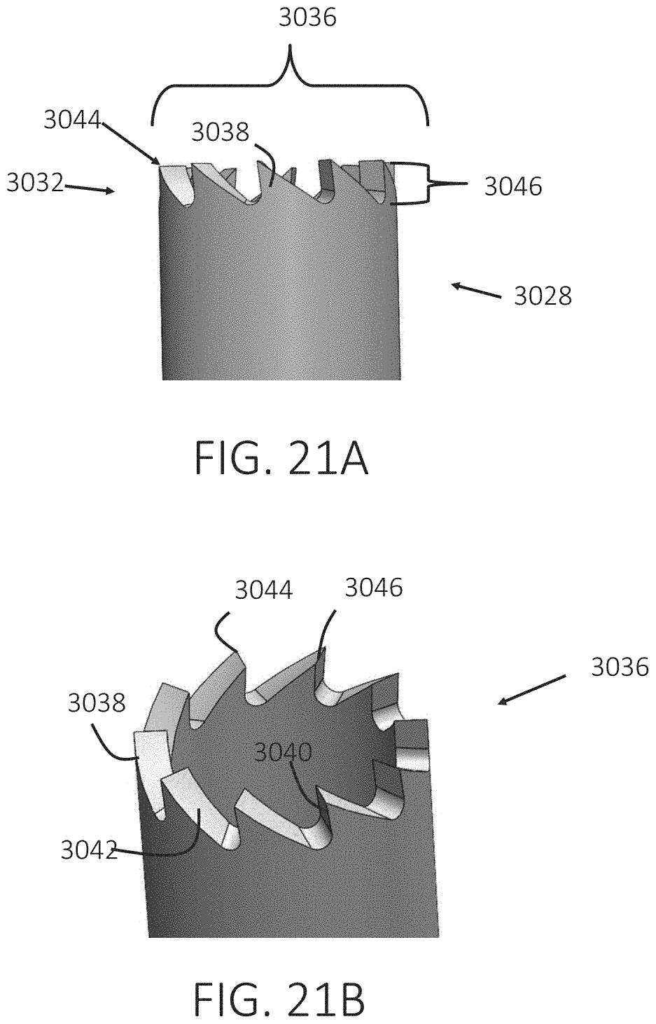

FIGS. 21A and 21B depict an embodiment of retrieval section 3028 having a plurality of teeth 3036 circumferentially disposed about leading-edge 3032 of retrieval section 3028. Each tooth 3038 includes a distinct cutting design form the embodiments depicted in the previous figures. Each tooth 3038 includes face 3040, back 3042, and point 3044, each tooth 3038 of the third cutting design has an aggressive angle of attack due to the positive rake angle. Furthermore, the gullet depth 3046 (the space between point and the valley of each tooth) and gullet area is increased by the positive angle of attach thereby increasing the amount of cellular material that can be retrieved while cutting.

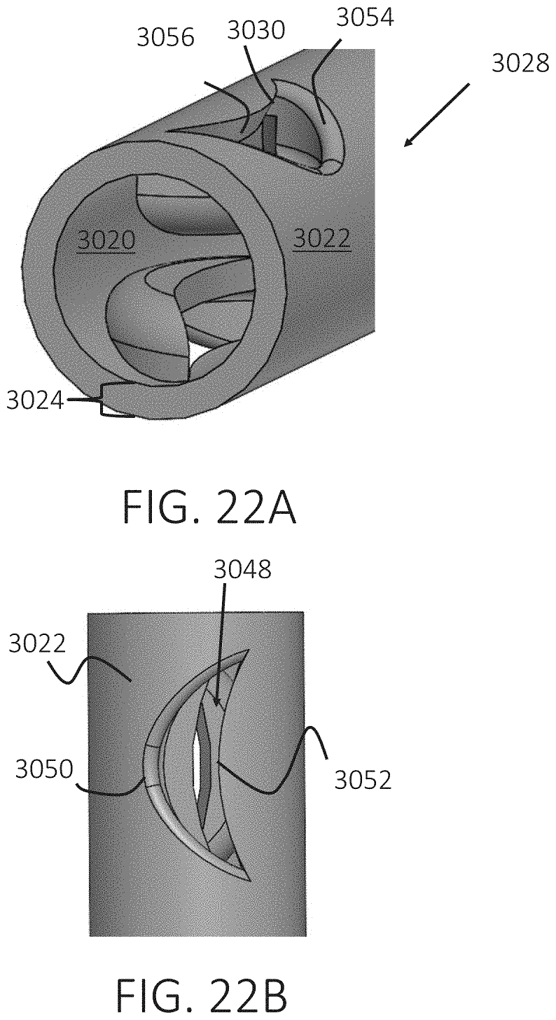

FIGS. 22A and 22B depict another embodiment of retrieval section 3028. It should be noted that FIGS. 22 do not depict the terminal end of biopsy needle 3010, which may have an angled or beveled shape to aid in the insertion of the needle and the cutting of tissue.

The embodiment of retrieval section 3028 as provided in FIGS. 22 includes a plurality of crescent-shaped cutting apertures 3048 disposed through lateral wall/body 3024. Each cutting aperture 3048 includes major aperture edge 3050 (the edge with the longer length) and minor aperture edge 3052 (the edge with the shorter length). In particular, major aperture edge 3050 comprises aperture wall 3054 extending between internal surface 3020 and exterior surface 3022 of elongated shaft 3012. Specifically, major aperture edge 3050 has a semi-circular shaped edge; however, alternative embodiments are contemplated having various geometrically shaped edges, such as square, linear, and triangular. Similarly, minor aperture edge 3052 includes beveled wall 3056 extending between internal surface 3020 and external surface 3022. However, alternative embodiments are contemplated having various geometrically shaped edges, such as square, linear, and triangular.

Cutting edge 3030 of beveled wall 3056 is formed at the intersection of external surface 3022 and wall 3056. In some embodiments, a line intersecting the midpoints of both major aperture edge 3050 and minor aperture edge 3052 is aligned perpendicular to central longitudinal axis 3014 of needle 3010. In some embodiments, the line intersecting the midpoints of both major aperture edge 3050 and minor aperture edge 3052 is non-parallel to central longitudinal axis 3014 of needle 3010. There orientations ensure that the rotation of needle 3010 about central longitudinal axis 3014 cut the adjacent tissue.

As retrieval section 3028 of biopsy needle 3010 is rotated about central longitudinal axis 3014, cutting edge 3030 engages with cellular material located within the biopsy area. Once the cellular material is dislodged from within biopsy area by cutting edge 3030, the cellular material is directed within bore 3026 via the beveled orientation of wall 3056 and preferably also a vacuum force created by the collection reservoir coupled with the proximal end 3016 of biopsy needle 3010. Additionally, multiple crescent cutting apertures 3048 can be disposed in distinct orientations or arrangements. Thus, regardless of how biopsy needle 3010 is manipulated, at least one cutting edge 3030 will engage the tissue of the biopsy area for collection.

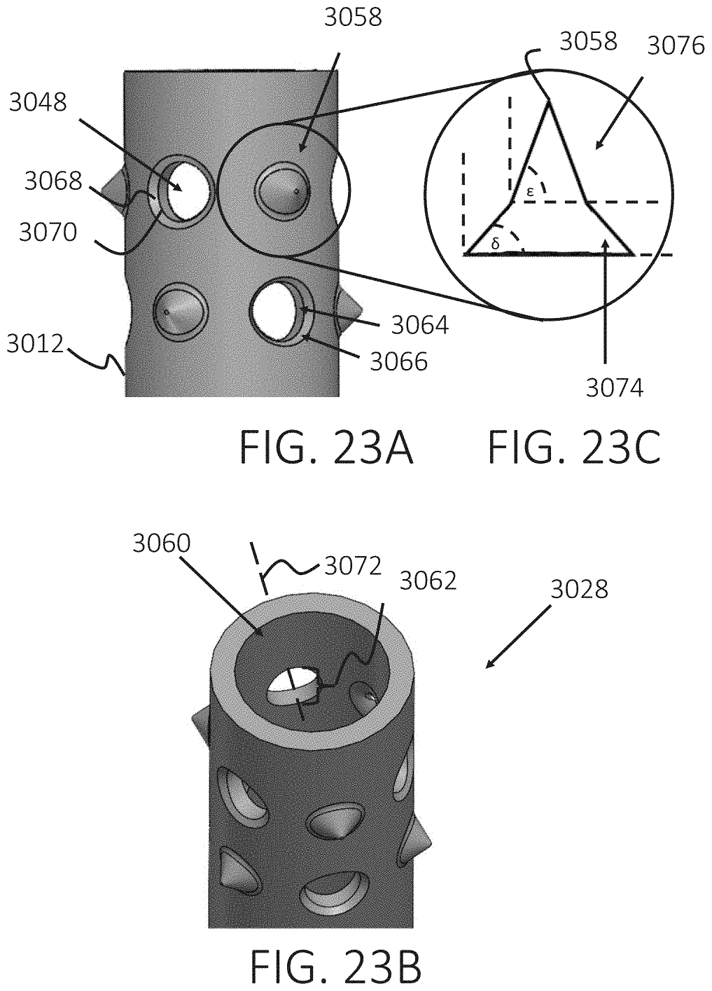

An embodiment of retrieval section 3028 of biopsy needle 3010 as shown in FIGS. 23 includes a plurality of circular cutting apertures 3048 and conical protrusions 3058 configured to cut and tear large portions of cellular material free during manipulation of biopsy needle 3010 within the biopsy area. Cutting apertures 3048 and protrusions 3058 may be randomly disposed of elongated shaft 3012 of biopsy needle 3010 or arranged in a pattern. As depicted in FIGS. 23A and 23B, a patterned embodiment includes alternating cutting apertures 3048 and protrusions 3058 in both a horizontal and vertical direction about elongated shaft 3012. During manipulation of biopsy needle 3010, dislodged cellular material may be collected within bore 3026 via cutting apertures 3048 and bore opening 3060.

Each cutting aperture 3048 is disposed through body 3024 of elongated shaft 3012 from internal surface 3020 to external surface 3022. More particularly, channel 3062 includes first portion 3064 and second portion 3066. First portion 3064 of channel 3062 includes a beveled edge and shares common boundary 3070 with second portion 3066. Channel 3062 includes channel axis 3072 disposed in an orthogonal relationship with central longitudinal axis 3014 of elongated shaft 3012.

Moreover, embodiments of cutting apertures 3048 disposed through body 3024 of retrieval section 3028 may include any other shape, size, or design of cutting apertures 3048 that is in line with any other embodiment of retrieval section 3028 disclosed herein.

Conical protrusions 3058 extend from external surface 3022 of elongated shaft 3012 from first protrusion end 3074 to second protrusion end 3076. First protrusion end 3074 of conical protrusion 3058 has a protrusion angle δ and second protrusion end 3076 has protrusion angle ε. Protrusion angle δ is a smaller angle than protrusion angle ε. Moreover, conical protrusion 3058 is configured to be in mechanical communication with the biopsy area and tears cellular material free, which is then collected through cutting aperture 3048 and/or bore opening 3060.

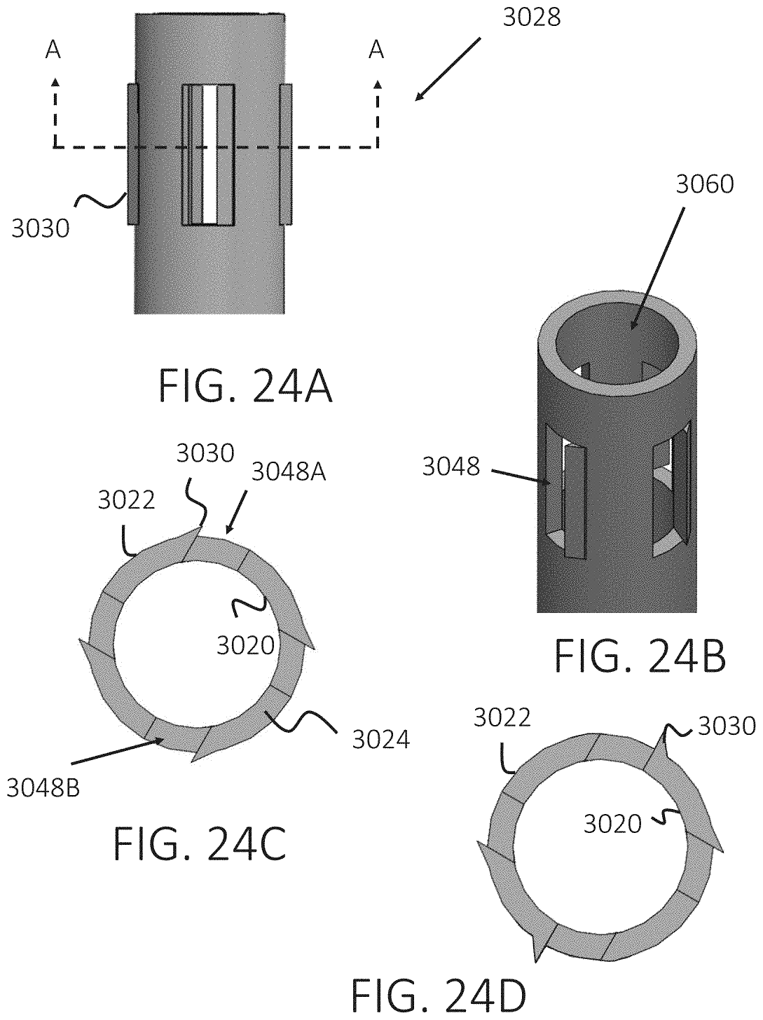

FIGS. 24A-24D depict an embodiment of retrieval section 3028 of the biopsy needle having a first pair of diametrically opposed cutting apertures 3048A and a second pair of diametrically opposed cutting apertures 3048B. Each cutting aperture 3048 is identical and similarly disposed within body 3024 of elongated shaft 3012 from external surface 3022 to internal surface 3020. In some embodiments, cutting apertures 3048 are not in pairs, but are equidistantly spaced about the circumference of the biopsy needle.

Each cutting aperture 3048 includes cutting edge 3030 extending outwardly away from external surface 3022. Each cutting edge 3030 is configured to engage with the tissue within the biopsy area, thereby dislodging the cellular material. Once dislodged, the cellular material is collected within bore 3026 via cutting aperture 3048 and/or bore opening 3060 disposed at distal end 3018 of biopsy needle 3010. Moreover, as depicted in FIG. 24D, a series of cutting edges 3030 extend outwardly from external surface 3022 and are configured to allow the retrieval section 3028 to cut the tissue within the biopsy area when retrieval section 3028 is rotated in both a clockwise and counterclockwise direction.

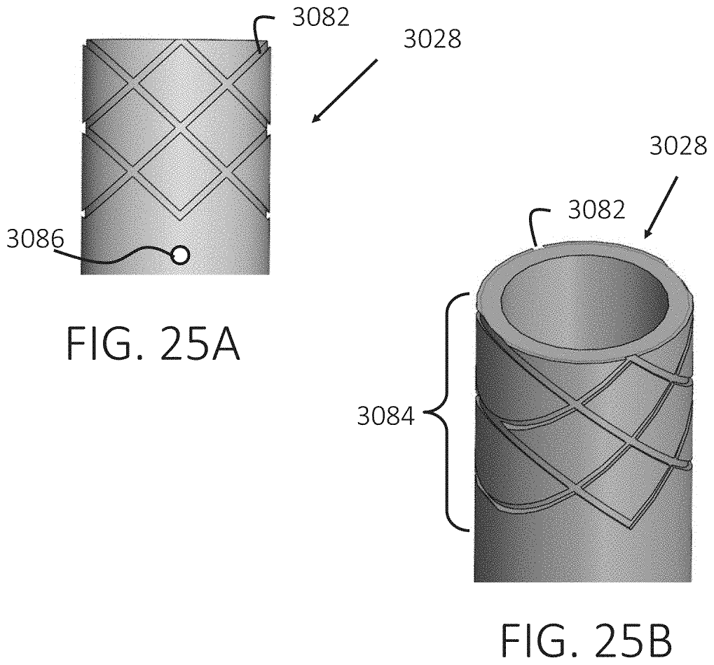

An embodiment shown in FIGS. 25A-25B includes retrieval section 3028 having chemically etched, or laser cut grooves 3082 disposed on elongated shaft 3012 of biopsy needle 3010. Grooves 3082 are configured to break cellular material free from within the biopsy area during manipulation of biopsy needle 3010. In particular, grooves 3082 may be straight, angled, crossed (as shown), or any other pattern that facilitates the collection of cellular materials. In an embodiment, retrieval section 3028 may include one or more cutting apertures disposed within the body of retrieval section 3028. In such embodiments, the cutting apertures may be in line with any other embodiment of retrieval section 3028 disclosed herein.

The material dislodged during manipulation of retrieval section 3028 is captured through bore opening 3060 and/or collection apertures 3086. Collection apertures 3086 may be disposed above, below, and or within knurling portion 3084 to facilitate the capture of dislodged cellular materials.

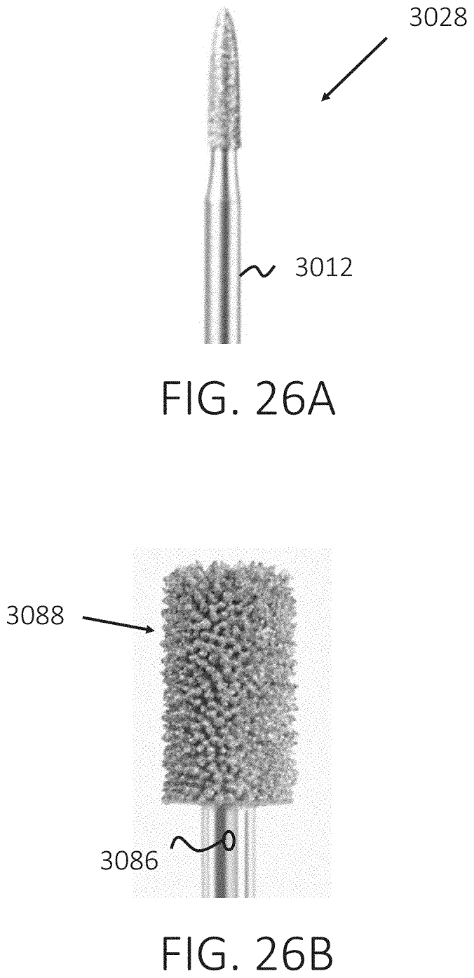

In some embodiments FIGS. 26A and 26B, retrieval section 3028 includes rough grinding surface 3088 disposed on external surface 3022 of retrieval section 3028. In some embodiments, rough grinding surface 3088 can be created by disposing foreign materials on the outer surface 3022 of elongated shaft 3012. Rough grinding surface 3088 is adapted to dislodge cellular material within the biopsy area by grinding cells away from the tissue of the biopsy area. Once cellular material is dislodged by grinding surface 3088, bore 3060 and/or collection apertures 3086 disposed throughout elongated shaft 3012 can be used to facilitate the collection of dislodged cellular materials.

In an embodiment, retrieval section 3028 may include one or more cutting apertures disposed within the body of retrieval section 3028. In such embodiments, the cutting apertures may be in line with any other embodiment of retrieval section 3028 disclosed herein.

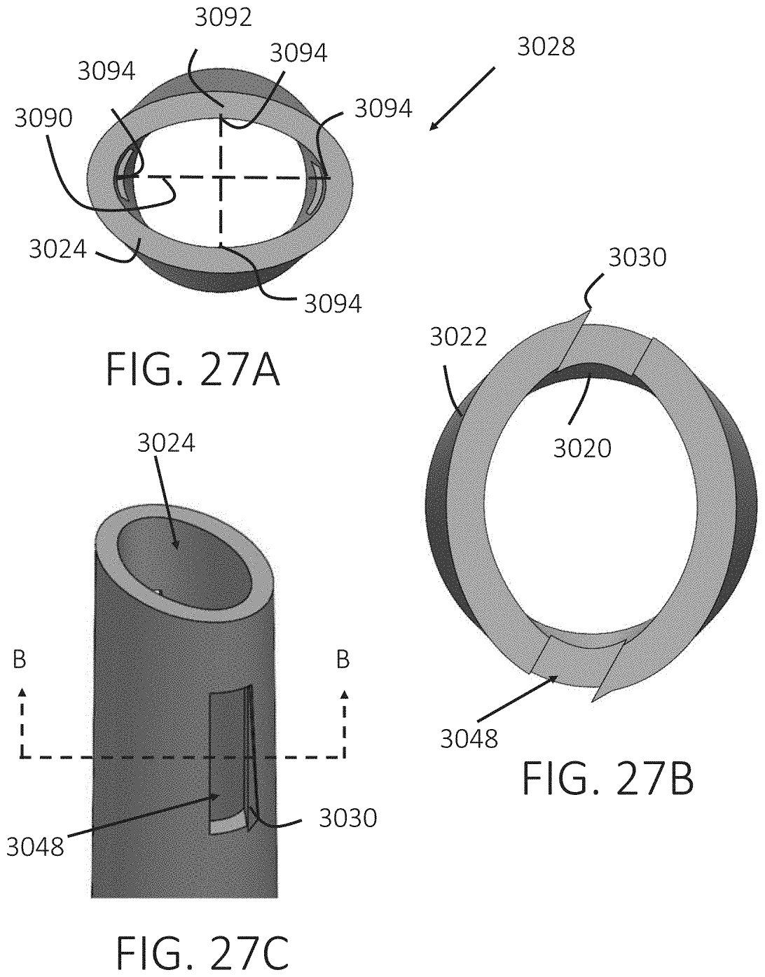

In an embodiment shown in FIGS. 27A-27C, retrieval section 3028 of biopsy needle 3010 has an angled tip, for example a 12-degree angle from the central longitudinal axis of the biopsy needle. In some embodiments, retrieval section 3028 has a cross-sectional geometry of an ellipse. The ellipse includes major axis 3090 and minor axis 3092 in an orthogonal relationship with major axis 3090.

Furthermore, retrieval section 3028 of includes a pair of diametrically opposed cutting apertures 3048 disposed at vertices 3094 of major axis 3090 of body 3024 of elongated shaft 3012 from internal surface 3020 to exterior surface 3022. Each cutting aperture includes cutting edge 3030 extending outwardly from external surface 3022 of body 3024. Cutting edge 3030 is configured to engage with the tissue within the biopsy area. Thus, when the biopsy needle is manipulated, cutting edge 3030 dislodges cellular material, which is collected within bore 3026 via cutting apertures 3048 and/or bore opening 3060.

Moreover, embodiments of cutting apertures 3048 disposed through body 3024 of retrieval section 3028 may include any other shape, size, or design of cutting apertures 3048 in line with any other embodiment of retrieval section 3028 disclosed herein.

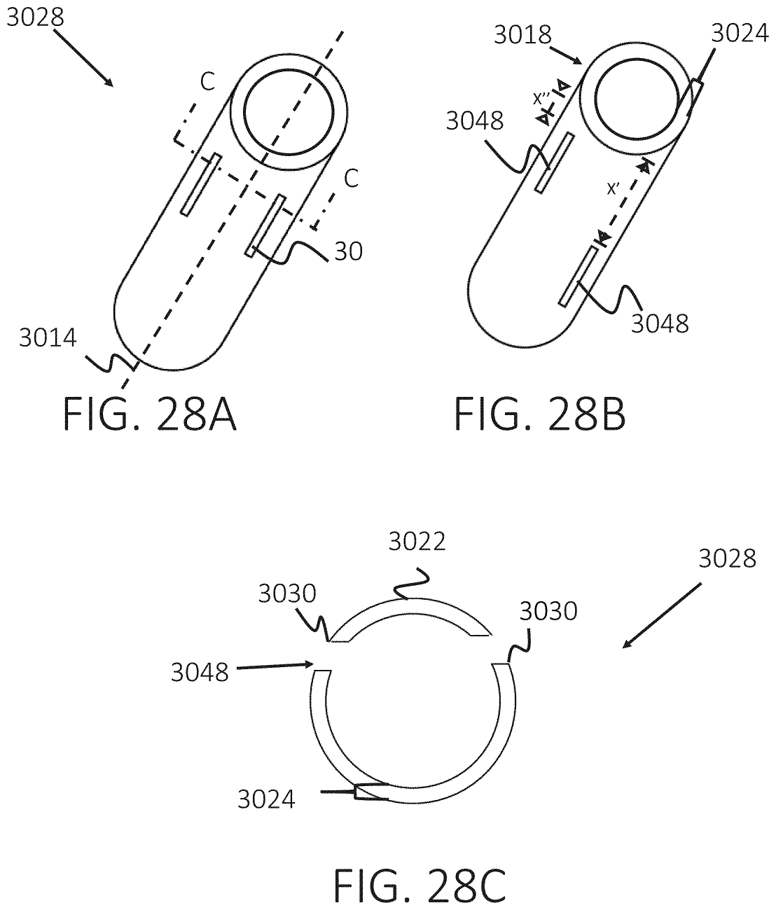

In some embodiments, as shown in FIGS. 28A-28C, retrieval section 3028 has a cross-sectional geometry of a circle; however, it is appreciated that various cross-sectional geometries may be provided depending on the specific needs of the biopsy needle during the procedure. Furthermore, cutting apertures 3048 are disposed within body 3024. Specifically, cutting apertures 3048 may be disposed from one another at the same distance from terminal end 29 (see FIG. 28A) or disposed at different distances (x′, x″) from terminal end 29 of retrieval section 3028 (see FIG. 28B).

Additionally, cutting edges 3030 are flush with and follow the curvature (i.e., circumference) of external surface 3022 of retrieval section 3028. In such embodiments, the manipulation of retrieval section 3028 laterally in an orthogonal relationship with axis 3014 forces tissues within the biopsy area in cutting apertures 3048. Thus, upon rotation of retrieval section 3028 about axis 3014 in either a clockwise or counterclockwise rotation, at least one of the cutting edges 3030 sheers off the tissue disposed within cutting aperture 3048 for sample collection.

Moreover, embodiments of cutting apertures 3048 disposed within body 3024 of retrieval section 3028 may include any other shape, size, or design of cutting apertures 3048 that are in line with any other embodiment of retrieval section 3028 disclosed herein.

Some embodiments, a depicted in FIG. 29 include multiple cutting apertures 3048 with each disposed on opposite sides of biopsy needle 3010. In some embodiments, outer lateral edge 3027 of channel 3062 is tangentially aligned with the interior surface 3011 of biopsy needle 3010.

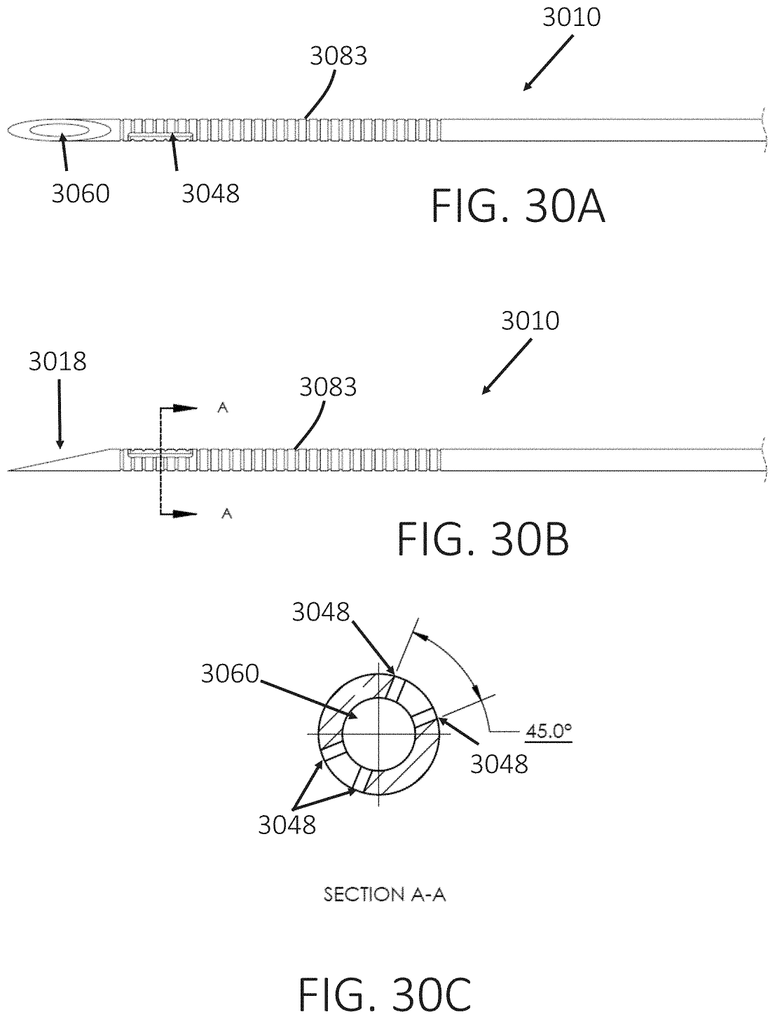

As illustrated in FIG. 30, some embodiments further include a plurality of annular grooves 3083. Grooves 3083 are configured to break cellular material free from within the biopsy area during manipulation of biopsy needle 3010. Similar to grooves 3082 in FIG. 25, grooves 3083 may be straight and perpendicular to the central longitudinal axis of needle 3010 as shown in FIG. 30 or they may be angled, crossed, or have any other pattern that facilitates the collection of cellular materials. In some embodiments, retrieval section grooves are located in the same general area as cutting apertures 3048, however, some embodiments may have grooves 3083 longitudinally offset from cutting apertures 3048.

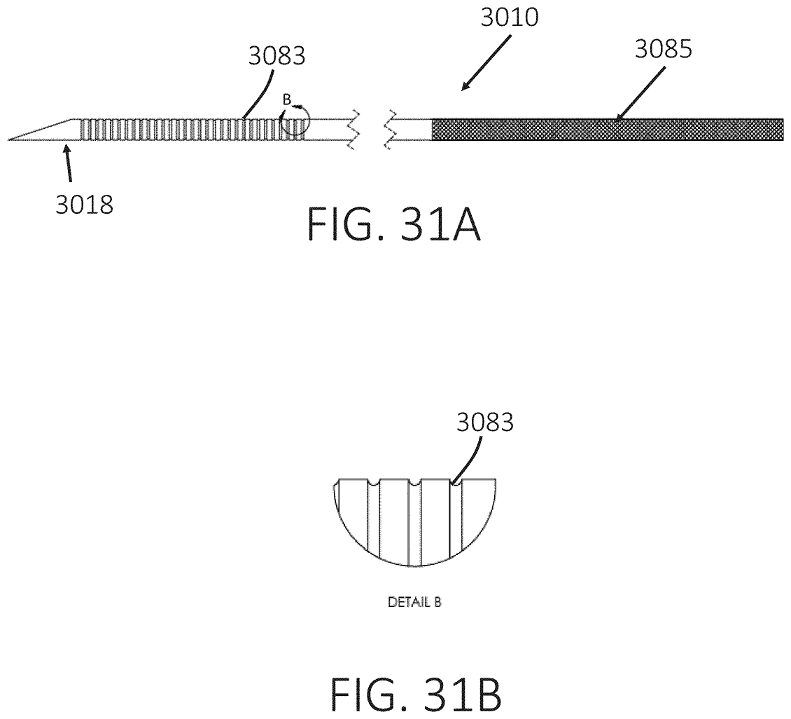

Moreover, some embodiments may include grooves 3083 without include cutting apertures 3048 as depicted in FIG. 31. In such embodiments, tissue enters bore channel 3060 in biopsy needle 3010 through distal end 3018.

Some embodiments include biopsy needle 3010 having a proximal section with knurling 3085 or other friction increasing features. The friction increasing features on the proximal portion aid in retaining a secure connection with needle 3010.

Referring back to FIG. 30C, the depicted embodiment illustrates the circumferential spacing of cutting apertures 3048 about needle 3010. The depicted embodiment includes two pairs of diametrically opposed cutting apertures 3048 with the closest cutting apertures 3048 circumferentially offset roughly 45 degrees. As depicted, the angular offset is from the furthest edges of the two adjacent cutting apertures 3048, however, alternative points on the two adjacent cutting apertures 3048 may establish the 45-degree separation.

In some embodiments, the circumferential spacing between two adjacent cutting apertures is between 5 and 90 degrees. In some embodiments, the circumferential spacing between two adjacent cutting apertures is between 5 and 180 degrees. It should also be understood that the circumferential spacing of cutting apertures may apply to the other embodiments disclosed herein.

### Method of Manufacturing

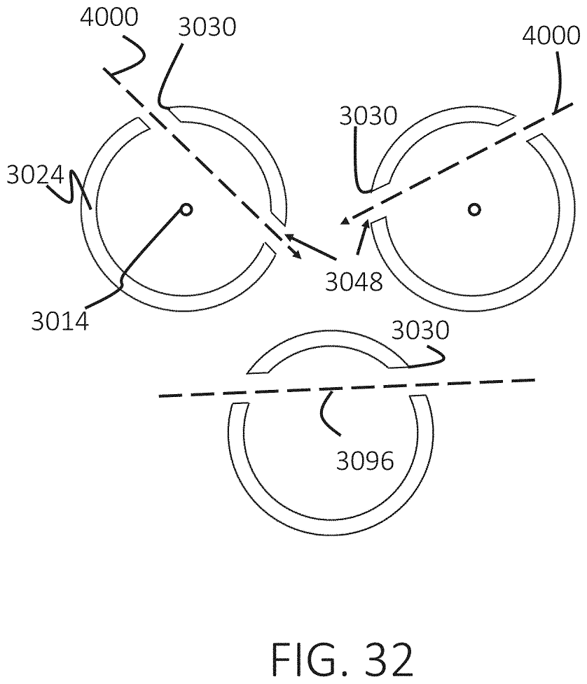

FIG. 32 depicts a method of manufacturing an embodiment of biopsy needle 3010 utilizing a novel methodology that results in biopsy needle 3010 having multiple cutting edges 3030 (or cutting apertures 3048). Each cutting edge 3030 is manufactured in pairs, such that no matter which direction biopsy needle 3010 is rotated about its central longitudinal axis 3014, cutting edge 3030 engages tissue within the biopsy area. In an embodiment, cutting edges 3030 are manufactured using a high-powered laser. In particular, the laser is directed toward body 3024 of elongated shaft 3012 at an angle along secant line 3096. In an embodiment, one or more laser cuts 4000 made be made along one or more secant lines 3096 to manufacture multiple cutting apertures 3048 within body 3024 of elongated shaft 3012, such as above or below previous laser cut(s).

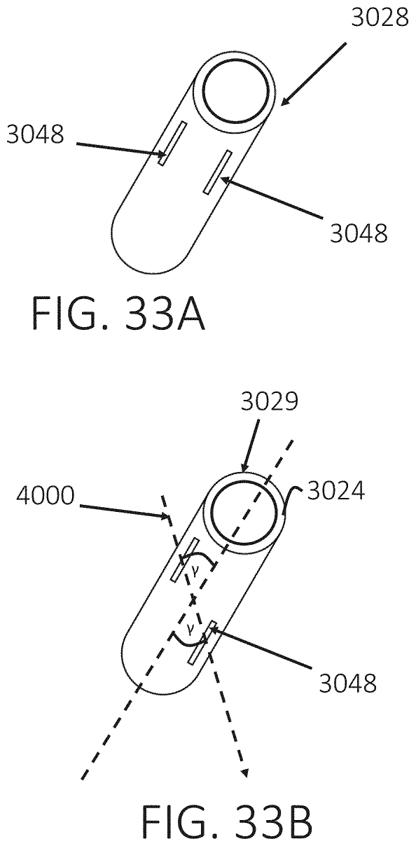

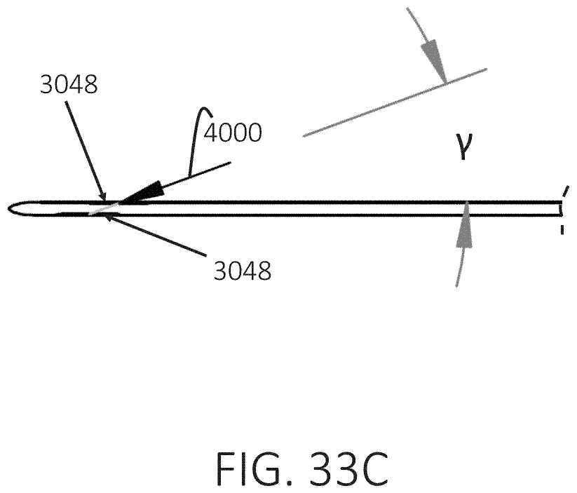

FIG. 33A depicts an embodiment of the novel method of manufacturing in which laser cuts 4000 are formed within body 3024 at the same distance from distal end 3018 of biopsy needle 3010. Alternatively, FIGS. 33B and 33C depict an embodiment of the novel method of manufacturing in which laser cuts 4000 are formed within body 3024 of elongated shaft 3012 at angle γ, such that cutting apertures 3048 are formed within body 3024 at different distances from distal end 29 of biopsy needle 3010. By manufacturing multiple cutting apertures 3048 of biopsy needle 3010 with a single pass of the laser, the overall manufacturing time of biopsy needle 3010 is significantly reduced.

While the particular device is herein shown and described in detail, it is to be understood that the subject matter which is encompassed by the present invention is limited only by the claims.

Components included in one embodiment can be used in other embodiments in any appropriate combination. For example, any of the various components described herein and/or depicted in the FIGS. may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

What is claimed:

1. A biopsy device, comprising:
a tissue collector having a distal end configured to be in fluidic communication with a biopsy needle;
a motor configured to rotate the biopsy needle;
the biopsy needle including:
a main body extending between a proximal end and a distal end;
a connector located at the proximal end, wherein the connector is configured to operably engage the motor, such that the motor can rotate the biopsy needle;
a cutting tip located at the distal end;
a central longitudinal axis extending between the proximal and distal ends;
a hollow interior extending through the main body and the connector, such that the hollow interior of the needle is in fluidic communication with the tissue collector;
a first cutting aperture disposed through the main body, wherein the first cutting aperture has a rectangular shape with a long end of the rectangular shape extending parallel to the central longitudinal axis of the biopsy needle;
a second cutting aperture disposed through the main body, wherein the second cutting aperture has a rectangular shape identical to the first cutting aperture with a long end of the rectangular shape extending parallel to the central longitudinal axis of the biopsy needle;
each of the first and second cutting apertures creating a channel with a central axis extending from an exterior surface of an interior surface of the main body, wherein the central axes of the channels are aligned; and
the first and second cutting apertures longitudinally spaced in a proximal direction from the distal end of the main body, such that there is a continuous portion of the main body between the first and second cutting apertures and the distal end of the main body.

2. The device of claim 1, wherein the motor further includes a rotational axis that is co-linear with the central longitudinal axis of the biopsy needle.

3. The device of claim 1, further comprising a belt coupling the motor to the biopsy needle to cause the biopsy needle to rotate under influence of the motor.

4. The device of claim 1, wherein a connector on the proximal end of the biopsy needle is a luer connector.

5. The device of claim 1, further comprising a valve in operable communication with a tissue evacuation passage extending between the biopsy needle and the tissue chamber, wherein the valve can lock vacuum in the tissue evacuation passage.

6. The device of claim 5, wherein the valve is a slide valve, the slide valve including a slidable actuator, wherein the slide actuator is configured to occlude the tissue evacuation passage when slid into a closed position.

7. The device of claim 1, further comprises:
at least one hollow fitting engaged with a needle connector, the hollow fitting comprising a body and a driven gear circumscribing the body and meshed with a drive gear coupled to the motor.

8. The device of claim 7, wherein the hollow fitting engaged with a needle connector rotates against an O-ring engaged with the drive housing.

9. The device of claim 1, further comprising at least driven and drive gears coupling the motor to the biopsy needle to cause the biopsy needle to rotate under influence of the motor.

10. The device of claim 1, wherein the biopsy needle is no larger than twenty-five (25) gauge.

11. The device of claim 1, wherein the first and second cutting apertures are diametrically opposed from each other about the main body of the biopsy needle.

12. The device of claim 1, wherein each of the cutting apertures are defined by a boundary circumscribing the aperture and the boundary is generally flush with an exterior surface of the main body.

13. The device of claim 1, wherein each cutting aperture is the same distance from the distal end of the biopsy needle.

14. The device of claim 1, wherein the first cutting aperture is longitudinally spaced from the second cutting aperture, such that the two cutting apertures are at different distances from the distal end of the biopsy needle.

15. The device of claim 1, wherein each cutting aperture includes a beveled channel wall extending between the interior and an exterior surface of the main body of the biopsy needle to direct tissue into the interior of the biopsy needle.

16. The device of claim 1, wherein each of the cutting apertures includes an outwardly, laterally extending flange relative to the central longitudinal axis of the biopsy needle.

17. The device of claim 1, wherein a portion of the biopsy needle proximal to the of the cutting apertures includes a plurality of annular grooves circumscribing an exterior surface of the biopsy needle.

18. The device of claim 1, further including:
a third cutting aperture disposed through the main body, wherein the third cutting aperture has a rectangular shape with a long end of the rectangular shape extending parallel to the central longitudinal axis of the biopsy needle;
a fourth cutting aperture disposed through the main body, wherein the fourth cutting aperture has a rectangular shape identical to the first cutting aperture with a long end of the rectangular shape extending parallel to the central longitudinal axis of the biopsy needle;
the third and fourth cutting apertures longitudinally spaced in the proximal direction from the distal end of the main body, such that the continuous portion of the main body is between the third and fourth cutting apertures and the distal end of the main body;
the third and fourth cutting apertures diametrically opposed from each other about the main body of the biopsy needle; and
the third cutting aperture is circumferentially spaced from the first cutting aperture by generally 45 degrees.

19. The device of claim 1, further including a plurality of annular grooves proximate the cutting apertures.

20. The device of claim 1, wherein the distal end of the biopsy needle includes a beveled tip.
